(19) United States
(12) Patent Application Publication
(10) Pub. No.: US 2011/0044964 A1
(43) Pub. Date: Feb. 24, 2011

(54) PLANT-BASED OMEGA CHEWABLE SUPPLEMENT

(75) Inventor: Judy Davis, San Clemente, CA (US)

Correspondence Address: THE ECLIPSE GROUP LLP, 6345 Balboa Blvd., Suite 325, Encino, CA, 91316, US

(73) Assignee: Hero Nutritionals, LLC, San Clemente, CA (US)

(21) Appl. No.: 12/781,200

(22) Filed: May 17, 2010

Related U.S. Application Data
(60) Provisional application No. 61/236,224, filed on Aug. 24, 2009.

(51) Int. Cl. A61K 38/43; A61K 36/00; A61K 31/202; C07C 57/12; A61P 43/00; A61K 31/201; C07C 57/03; A61P 17/18

(52) U.S. Cl. 424/94.1; 424/725; 514/560; 554/224

(57) ABSTRACT

A chewable composition for the oral delivery of plant-based omega fatty acid compounds. The chewable composition includes a delivery vehicle and an active ingredient incorporated therein. The delivery vehicle may include an organic or in-organic gummy candy made from a binding agent, sweeteners, flavoring, and coloring. The active ingredient may include a plant-based omega fatty acid to provide a desired effect on the user. The delivery vehicle may also include any combination of nutraceuticals, vitamins, minerals, antioxidants, soluble and insoluble fiber, herbs, plants, probiotics, prebiotics, antioxidants, amino acids, digestive enzymes, proprietary dietary supplements, or any other health promoting ingredient.

### RELATED APPLICATIONS

This application claims priority from U.S. Provisional Application No. 61/236,224, filed on Aug. 24, 2009, titled PLANT-BASED OMEGA-3 CHEWABLE SUPPLEMENT, which application is incorporated in its entirety by reference in this application.

### BACKGROUND OF THE INVENTION

1. Field of the Invention

The invention relates generally to a chewable dietary supplement, and more particularly to a chewable composition for the oral delivery of plant-based omega compounds and a method for manufacturing the same.

2. Related Art

Research supports the cholesterol-lowering benefits of eating fatty fish because of its high levels of fatty acids. Fatty acids are acids produced when fats, such as those found in oils and other fats that make up different foods, are broken down. While we tend to think of all forms of “fats” as bad or unhealthy, fatty acids are actually essential for optimum health. The human body needs fatty acids for several reasons: fatty acids help move oxygen through the bloodstream to all parts of the body; they aid cell membrane development, strength, and function; they produce chemicals that regulate blood clotting and blood pressure; and they are necessary for strong organs and tissue.

Fatty acids are long-chain (monounsaturated, polyunsaturated, and saturated) acids found mainly in plants and fish. Fatty acids are organic, meaning that they contain both carbon and hydrogen molecules. Chemically, fat molecules are made up of four parts: a molecule of glycerol on one end of the molecule chain, and three molecules of fatty acids on an opposite end of the molecule chain. Each fatty acid consists of a hydrocarbon chain with a carboxyl group at one end. Fatty acids are typically referred to as omega fatty acids and the number following the term “omega-” represents the position of the first double bond, counting from the terminal methyl group on the molecule.

Of these fatty acids, a subset called essential fatty acids (EFAs) are necessary fats that cannot be synthesized or produced by the human body and, therefore, must be obtained through the human diet. EFAs are long-chain polyunsaturated fatty acids that are classified into two families: omega-3 fatty acids and omega-6 fatty acids. A third family, omega-9 fatty acids, are also necessary, yet “non-essential” fats because the body produces a modest amount of these fats on its own.

Omega-3 fatty acids are a family of unsaturated fatty acids having in common a final carbon-carbon double bond in the n-3 position; i.e., the third bond from the methyl end of the fatty acid. There are eight members in this family of fatty acids, with the three most important being a-linolenic acid (ALA), eicosapentaenoic acid (EPA), and docosahexaenoic acid (DHA). As the health benefits of the omega-3 fatty acids have become more widely known, DHA and EPA being the best known, more and more food products are being enriched with omega-3 fatty acids.

There have been numerous studies made of the health benefits of omega-3 fatty acids. These benefits can be classified as relating generally to heart, joint, and brain health. There are studies that show evidence of cardiovascular benefits, such as reducing cholesterol and triglycerides in the blood stream and maintaining optimum blood pressure. Additionally, omega-3 fatty acids are believed to give relief from joint pain and migraines and may help lower the risk of cancer, Alzheimer's disease, depression, attention deficit-hyperactivity disorder (ADHD), and a host of other diseases.

Pregnant women, in particular, have an increased need for omega-3 fatty acids. Studies have shown that fatty acids are needed for fetal growth, brain development, learning, and behavior. Thus, lactating women are encouraged to increase their intake of fatty acids since infants receive their essential fatty acids through breast milk As more research is undertaken, people become more aware of the possible health benefits of omega-3 fatty acids and there is added incentive to supplement a person's diet with omega-3 fatty acids. Some medical experts recommend at least 500 mg per day of combined EPA and DHA.

In addition to omega-3 fatty acids, other fatty acids such as omega-6 and omega-9 fatty acids are also known to provide health benefits. Omega-6 fatty acids are a family of unsaturated fatty acids having in common a final carbon-carbon double bond in the n-6 position; i.e., the sixth bond from the methyl end of the fatty acid. There are nine members in this family of fatty acids, with the two most important being linoleic acid (LA), which may be found in soybean oil, corn oil, safflower oil, peanut oil, cottonseed oil, and rice bran oil; and Arachidonic acid (AA), which may be found in certain meats, eggs, and dairy products. Like omega-3 fatty acids, omega-6 fatty acids are polyunsaturated fats that cannot be produced in the human body. Thus, omega-3 and omega-6 fatty acids are classed as essential fatty acids (EFAs) to the human diet that can only be obtained by consuming foods such as meat, poultry and eggs, as well as nut and plant-based oils.

Omega-9 fatty acids are a family of unsaturated fatty acids having in common a final carbon-carbon double bond in the n-9 position; i.e., the ninth bond from the methyl end of the fatty acid. There are five members in this family of fatty acids, with the two most important being oleic acid (OA), which is commonly found in olive oil, canola oil, and other monounsaturated fats; and erucic acid (EA), which can be extracted from rapeseed, wallflower seed, and mustard seed.

Unlike omega-3 and omega-6 fatty acids, omega-9 fatty acids may be produced by the body, but are most beneficial when they are obtained through foods. Studies have shown that omega-9 fatty acids may help lower the risk of cardiovascular disease and stroke. Because omega-9 fatty acids have been shown to increase HDL (“good”) cholesterol and decrease LDL (“bad”) cholesterol, they help eliminate plaque buildup in the arteries, which may cause heart attack or stroke.

The most widely available source of fatty acids, namely EPA and DHA, is cold-water oily fish, such as mackerel, herring, tuna, salmon, anchovies, and sardines. Most people in the Western world do not get enough omega fatty acids in their diet, especially those with low fish diets. Therefore, there is a need to supplement the human diet with the omega fatty acids.

The omega fatty acids in the form of fish oil are safe for most people when used appropriately. However, fish oil can have a “fishy” aftertaste and can also cause belching, nosebleeds, nausea, and loose stools. A major concern with fish oil is that because fish do not produce the omega fatty acids themselves but accumulate it in their bodies from the ingestion of microalgae and seaweed, the fish oil may contain traces of heavy metals, in particular, mercury and other contaminants. Thus, a need exists for a safe, easily digestible, and palatable dietary supplement that will directly supply a sufficient dosage of the omega fatty acids to the human body, without the drawbacks of fish oil mentioned above.

### SUMMARY

An edible, digestible composition is provided that includes a chewable delivery system in the form of an organic or non-organic gummy candy and a predetermined dosage of plant-based omega fatty acids. By ingesting the gummy candy, the consumer is able to directly supply his/her body with the required dietary intake of omega fatty acids.

According to one implementation, the delivery system includes a delivery vehicle in the form of a gummy candy, and an omega fatty acid as the active ingredient of the gummy candy. The gummy candy may include a binding agent, a sweetener, flavoring and coloring, and a polishing agent. For example, in one implementation, the gummy candy may include gelatin, sucrose, corn syrup, citric acid, lactic acid, natural colors, natural flavors, fractionated coconut oil, and carnauba wax.

In other implementations, the delivery vehicle may include other digestible forms such as tablets, capsules, or solid (hard) or chewable candies.

In addition to the omega fatty acid, the delivery system may also include a blend of dietary supplements. These supplements may include nutraceuticals (i.e., extracts of food purported to have a medicinal effect on human health) such as botanical and herbal extracts and antioxidants, or any combination of food supplements such as vitamins, minerals, soluble and insoluble fibers, herbs, plants, probiotics, prebiotics, amino acids, digestive enzymes, or any other health promoting ingredient.

Other devices, apparatus, systems, methods, features and advantages of the invention will be or will become apparent to one with skill in the art upon examination of the following FIGURE and detailed description. It is intended that all such additional systems, methods, features and advantages be included within this description, be within the scope of the invention, and be protected by the accompanying claims.

### BRIEF DESCRIPTION OF THE FIGURES

The invention may be better understood by referring to the following FIGURE. The components in the FIGURE are not necessarily to scale, emphasis instead being placed upon illustrating the principles of the invention. In the FIGURE, like reference numerals designate corresponding parts throughout the different views.

FIG. 1 is a flow diagram that illustrates one example method of manufacturing a chewable supplement of the present invention.

### DETAILED DESCRIPTION

The present invention relates to a plant-based omega fatty acid delivery system, in particular, a digestible delivery system that is designed to enhance the delivery of plant-based omega fatty acids. The delivery system includes a primary active ingredient (a dosage of plant-based omega fatty acids) to provide the desired effect, and a delivery vehicle (e.g., a gummy candy) to contain the active ingredient for delivery.

The primary active ingredient of the present invention may include, for example, plant-based omega-3 fatty acids. Plant sources for the omega fatty acids include flax (also known as linseed), chia, kiwi fruit, ligonberry, purslane, walnuts, and canola and soybean oil. Another plant-based source of omega-3 fatty acids is marine algae.

The primary active ingredient, i.e., the plant-based omega fatty acid, is delivered in a delivery vehicle that is palatable and easy to swallow. In one implementation, the delivery vehicle is chewy or gummy-like to facilitate ingesting all of the active ingredients. The delivery vehicle may include a binder, sweeteners, coloring, and flavoring. In one implementation, the delivery vehicle may include glucose syrup, natural cane juice, gelatin, citric acid, lactic acid, natural colors, natural flavors, fractionated coconut oil, and carnauba wax.

In addition to fatty acids, the delivery system may also include any combination of vitamins, minerals, antioxidants, soluble and insoluble fibers, herbs, plants, probiotics, prebiotics, nutraceuticals, amino acids, digestive enzymes, or any other supplements digested to promote the heath and well-being of a person. Inclusion of any of these dietary supplements will be dependent in part on their compatibility with the plant-based omega fatty acids.

Manufacturing of Delivery System

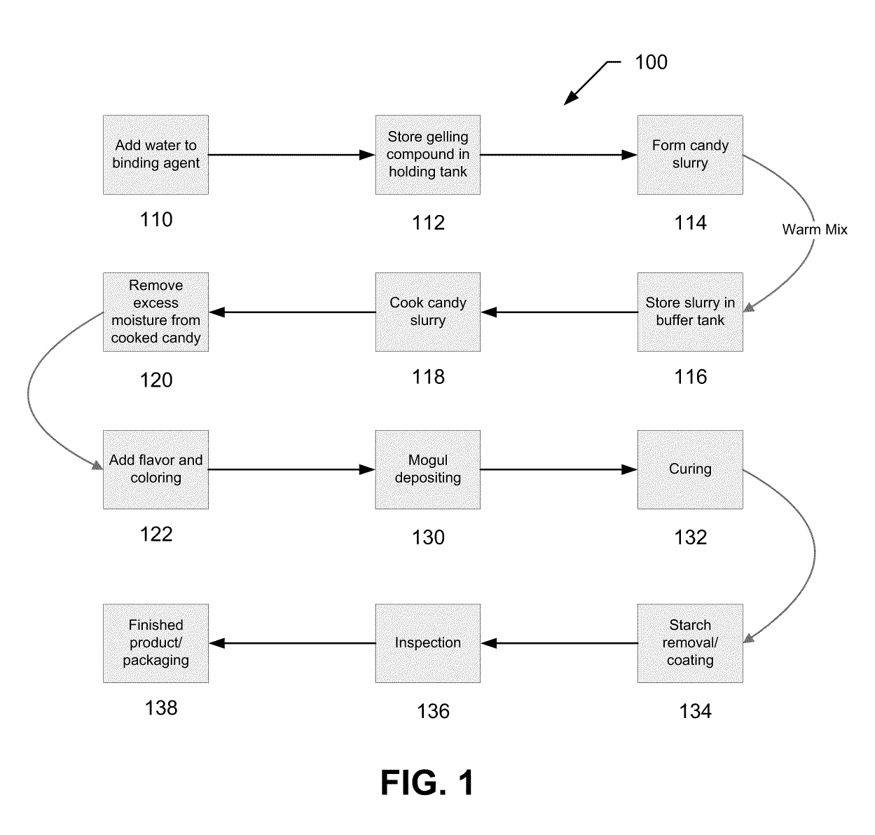

Turning now to FIG. 1, a method 100 for manufacturing a delivery system of the present invention is disclosed. In general, the method of manufacturing involves three main phases: (i) compounding (i.e., mixing) and storing; (ii) batching and cooking; and (iii) depositing and curing.

The first phase of compounding and storing begins with step 110, where water and a binding agent are mixed in a mixing tank to form a gelling compound. In one implementation, the mixing tank may include a 1,000 gallon stainless steel planetary mixer, a scrape surface mixer, a holding tank with an agitator, or any other suitable mixer. During production, water and the binding agent are continuously mixed in the mixing tank. The gelling compound is continuously turned in the tank by an agitator to keep the binding agent suspended in water (i.e., to prevent the binding agent from settling on the bottom of the mixing tank). In one implementation, approximately 6,000 lbs to 8,000 lbs of gelling compound may be produced in 8 hours.

The gelling compound may include cold, warm, or hot water. However, warm or hot water may be used to reduce the hydration time of the gelling compound. For example, about 250 lbs of gelatin mixed with about 250 lbs of warm water may reach a homogenous mixture in about 10 minutes. The hydration rate of the gelling compound may also vary according to the speed of the agitator. As used herein, “hydration time” or “hydration rate” refers to the time it takes water to hydrate the binding agent.

As used herein, the term “homogeneous” refers to a composition, solution or mixture whose elements are substantially uniformly dispersed in each other. For example, a homogenous composition may include two or more compounds or elements which are substantially uniformly dispersed within each other.

The binding agent may include gelatin, pectin, food starch, carrageenan, gum, or any other suitable binder, or combination thereof. For example, the binding agent may include gelatin products produced from animal sources such as beef or pork, or any other suitable gelatin product. Such products may include GELITA® Gelatine products sold by Gelita USA, Inc.

Examples of pectin products may include high (methyl) ester or low (methyl) ester pectin products made from fruit sources, such as apples, apricots, carrots, citrus fruits, or any other suitable pectin product. Such products may include, for example, UNIPECTIN® HM-pectin and/or UNIPECTIN® LM-pectin products.

Examples of starch ingredients may include corn starch, rice starch, potato starch, starch derivatives, and the like.

Examples of carrageenan ingredients may include kappa (κ) carrageenans sold under the Gelcarin® brand, or lambda (λ) carrageenans sold under the Viscarin® brand, both available from FCM Corporation.

Depending on the binding agent used, the gelling compound may include, for example only, one of the following formulations:

TABLE A
GELLING COMPOUND FORMULA

| Binding Agent | Binding Agent (% by weight) | Water (% by weight) |
|---|---|---|
| gelatin | 50% | 50% |
| pectin | 2%-3% | 97%-98% |
| starch | 7%-10% | 90%-93% |
| pectin/starch | 8%-10% | 90%-92% |
| | (1%-2% pectin/7%-8% starch) | |
| gelatin/starch | 7%-9% | 93%-91% |
| | (1%-2% gelatin/6%-7% starch) | |
| carrageenan | 2%-5% | 95%-98% |

In one implementation, a buffer, such as hydroxides, carbonates, citrates and phosphates, and mixtures thereof and their salts (e.g., sodium bisulfate or sodium citrate), may be mixed into the gelling compound to regulate the pH of the mixture. In one implementation, the gelling compound may contain approximately 0.01 to 0.03% buffer by weight, or any other suitable amount. The pH of the mixing tank may be adjusted to a range from about 3.2 to about 4.0 to provide adequate gelation and to ensure that the gelling compound does not become unstable (i.e., acidic) during mixing. In implementations where the binding agent includes starch, a buffer may not be necessary to balance the pH of the compound because starch is generally a stable organic compound.

At step 112, the gelling compound may be filtered through a fine mesh, to remove particulates in the compound, and stored in a holding tank. In one implementation, about 140 lbs to 190 lbs of gelling compound may be delivered from the mixing tank to the holding tank every 5 to 10 minutes. The filter may be a 0.034 inch stainless steel basket strainer. The holding tank may be a 1,500 gallon stainless steel tank. In some implementations, the holding tank may include a moderate agitator (e.g., mixing blades) to mix the compound and prevent the binding agent from settling on the bottom of the holding tank during storage.

From the holding tank, approximately 125 lbs to 185 lbs of gelling compound may be delivered to a mixing vessel at step 114, every 5 to 10 minutes, for example. In one implementation, the mixing vessel may be a 5,000 gallon stainless steel planetary mixer. In other implementations, the mixing vessel may be a scrape surface mixer, a holding tank with an agitator, or any other type of suitable mixer.

In the mixing vessel, water, additives, the active ingredients, and additional supplements, if any, may be added to the gelling compound to form a candy slurry mixture. For example, the additives may include sodium citrate, sweeteners such as sugar (also referred to herein as sucrose or natural cane juice) and/or syrup (e.g., corn, glucose, rice, tapioca), and corn starch, in liquid and/or powdered form.

The active ingredient of the present invention may include any combination of plant-based omega fatty acids. For example, these plant-based fatty acids may include omega-3 fatty acids, omega-6 fatty acids, omega-9 fatty acids, or any other suitable plant-based omega fatty acids, or combinations of the same.

Plant sources for the omega-3 fatty acids may include flax (also known as linseed), chia, kiwi fruit, ligonberry, purslane, walnuts, cranberry seed, canola, and soybean oil. Of theses sources, flax is the most widely available. Flaxseed oil consists of approximately 55% ALA, which is converted in the body to EPA and then to DHA. Another plant-based source of omega-3 fatty acids is marine algae, from which DHA may be extracted.

Plant sources of omega-6 fatty acids may include leafy vegetables, seeds, nuts, grains and vegetable oils (corn, safflower, soybean, cottonseed, sesame, sunflower). Plant sources for the omega-9 fatty acids may include olive oil, canola oil, sunflower oil, and almonds, for example.

As for the dosage, because omega fatty acids are macronutrients, they are not assigned recommended daily allowances. One recommendation is 500 mg per day of combined EPA and DHA for a healthy person. Because ALA is converted in the body at an efficiency rate of 5-10%, the dosage of ALA, in the form of flaxseed oil, can be considerably higher. By way of example only, a singe piece of gummy candy may have 1-2 grams of flaxseed oil. Generally, the dosages in each gummy candy should be relatively low, allowing the consumer to determine the proper dosage by starting with a lower dosage and increasing if necessary, i.e., by trial and error.

In addition to the active ingredients, additional dietary supplements may be added to the slurry mixture. These supplements may include vitamins, minerals, herbs, plants, probiotics, prebiotics, fibers, amino acids, digestive enzymes or any other supplements digested to promote the heath and well-being of a person. For example, the supplements may include, but not be limited to, any of the following:

Vitamin B1 (Thiamine)

Vitamin B2 (Riboflavin)

Vitamin B3 (Niacinamide)

Vitamin B5 (Pantothenic Acid)

Vitamin B6 (Pyridoxine HCL)

Vitamin B12

Biotin

Folic Acid

Vitamin C (Ascorbic Acid/Activated C)

Calcium

Carotine

Chromium

Choline

Copper

Magnesium

Zinc

Protein

Pomegranate

Inositol

Vitamin D (Cholecalciferol)

Vitamin E (Acetate)

Ginseng

Iron

Vitamin K (Phytonadione)

St. John's Wort

The above list of raw materials are not exhaustive, but are provided for illustrative purposes only. The length of a list of all available supplements that may be utilized in the chewable supplement of the invention is too lengthy to provide.

In one implementation, the candy slurry mixture may contain approximately 70% to 85% sweetener by weight, while the remaining approximately 15% to 30% of the slurry (by weight) may contain the gelling compound and additives. More particularly, the slurry may contain approximately 19% water, 2% sodium citrate, 30% natural cane juice, 45% corn syrup, 2% supplements, and 2% primary active ingredient by weight. In most implementations, the candy slurry may reach a homogeneous mixture in about 5 to 10 minutes.

The ingredients described above and their compositions are provided by way of example only. Without departing from the spirit and scope of the present invention, the ingredients and the composition of the candy slurry may vary based on the type of formulation desired.

Prior to production, the sugar and syrup additives may be stored in bulk tanks. In one implementation, the syrup may be stored in a holding tank at a temperature of approximately 75° F. In the holding tank, the syrup may be irradiated by ultraviolet light to remove any contaminants in the syrup. The syrup may include high fructose corn syrup (e.g., HFCS-42, HFCS-55, or HFCS-62), glucose syrup, rice syrup, tapioca syrup, or any other suitable liquid sweetener or combination thereof. During production, the syrup may be administered to the mixing vessel manually or by automation.

Similarly, prior to production, sugar in granular form may be stored in a holding tank. During production, sugar may be fed through an automated feed system that filters the sugar to remove sediments, weighs the sugar, and delivers a desired quantity of sugar to the mixing vessel. In other implementations, sugar may be added to the mixing vessel manually.

Turning back to FIG. 1, from the mixing vessel, the candy slurry is processed through a magnetic device, which removes particulates from the slurry, and stored in a storage buffer tank at step 116. In one implementation, the magnetic device may be a finger magnet or any other suitable magnetic device. The storage tank may be a 5,000 gallon stainless steel industrial holding tank. In other implementations, the holding tank may include a moderate agitator to suspend the active ingredients in the candy slurry. Prior to reaching the storage buffer tank, the candy slurry may be heated through a series of heat exchangers to a temperature of approximately 150° F. to 180° F.

In one implementation, the storage buffer tank may receive the candy slurry mixture from the mixing vessel at a mass flow rate of approximately 15 lbs/s to 20 lbs/s, and maintain the slurry at a temperature of approximately 150° F. to 200° F. Simultaneously, the warm candy slurry may be continuously fed from the storage buffer tank to a static cooker at mass flow rate of approximately 10 lbs/s to 15 lbs/s, by way of example only.

In the next phase of batching and cooking, at step 118 the candy slurry mix is received by the static cooker and cooked at a temperature of approximately 220° F. to 260° F. for approximately 30 sec. to 60 sec., until the slurry is gelatinized (i.e., dehydrated). In one implementation, the static cooker may be a 2,500 gallon high pressure steam jacketed kettle, a vacuum pressure cooker, or any other suitable cooker. In the static cooker, moisture is evaporated out of the candy slurry as the slurry is boiled to a temperature of approximately 250° F. After about a minute of boiling, the slurry may consist of about a 65 to 75 brix solution.

As used herein, the term “brix” refers to the dissolved sugar-to-water ratio of a liquid or gel. For example, as described above, after boiling, the slurry mixture may consist of approximately 65 grams to 75 grams of sugar and approximately 25 grams to 35 grams of water per 100 grams of solution.

After the candy slurry is cooked, a vacuum is applied to the candy at step 120. In one implementation, the pressure cooker may include a vacuum apparatus. In another implementation, the cooked candy may be delivered to an industrial vacuum chamber or any other suitable enclosure.

At the vacuum step 120, moisture is drawn from the cooked candy by suction pressure. In one implementation, a vacuum of approximately 40 psi to 50 psi applied to the candy stock for approximately 15 sec. to 30 sec. may draw out approximately 2% to 5% water by weight. However, the pressure of the vacuum and the vacuum rate will vary according to the capabilities and size of the vacuum apparatus. At this juncture, the cooked candy may have a brix of approximately 67 to 80, and a pH of approximately 2.8 to 4.0, for example.

From the vacuum, the cooked candy is filtered through a strainer into a trough-like apparatus, commonly known as a dosier. In other implementations, the process of adding flavoring and coloring to the cooked candy, as described below, may be performed by an apparatus incorporated within a starch molding machine; a starch molding machine is described in detail below.

At this point in the manufacturing process, the cooked candy mainly consists of a clear gelatinized composition. To obtain a desired color and taste, coloring and flavoring may now be added to the cooked candy.

At step 122, the cooked candy may be passed through the dosier. In the dosier, water, flavoring, coloring, and food grade acid may be added to the cooked candy to enhance the candy's taste. For example, flavoring such as artificial and/or natural flavoring (e.g., fruit concentrate) may be added to the cooked candy to give the candy a desired flavor. To balance the flavor, food grade acid may be added to the cooked candy. Such food acids may include citric acid, malic acid, lactic acid, adipic acid, fumaric acid, tartaric acid, or any other suitable food acid or combinations thereof. In one implementation, the flavoring, coloring, and acid may be continuously added to (e.g., dripped on) the cooked candy as the candy moves through the dosier to the mogul machine.

The amount of flavoring, coloring, and acid administered to the cooked candy may vary according the volume of cooked candy passing through the dosier and the desired candy formulation. For example, in one implementation, approximately 1% to 2% flavoring by weight and approximately 0.01% to 0.03% acid by weight may be added to the cooked candy composition. However, the amount of acid and flavoring added to the cooked candy formulation must be balanced to insure that the cooked candy will taste good. So, depending on the formulation, more flavoring and less acid may need to be added to the cooked candy for bitter formulations. For instance, to mask the flavor of the fatty acid, a flavoring agent such as strawberry flavor or cherry flavor may be added to the mixture. The additional flavor would be adjusted based upon the fatty acid dosage. In some instances, only food acid instead of flavoring may be added the cooked candy.

In addition to food acid, coloring and titanium dioxide may be added to the cooked candy formulation in the dosier. Coloring may be added to give the candy a desired color or colors. Coloring may include natural coloring such as black carrot, annatto, turmeric, and purple berry concentrate, or artificial coloring such as yellow 5, red 3, and blue 1, or any combination thereof.

Titanium dioxide may be added to the candy to provide sheen. Titanium dioxide may also stabilize the cooked candy formulation so the coloring does not bleed when it is handled, packaged, or stored.

In the final phase of depositing and curing, after the cooked candy is passed through the dosier, the candy may be sent to a starch molding machine at step 130. In one implementation, the starch molding machine may include a mogul machine (simply referred to as a “mogul”). A mogul is a starch molding machine that automatically performs the multiple tasks involved in making gummy candy.

Gummy candy is produced in the mogul by a continuous process. To start the process, the cooked candy, or gummy stock, is deposited by depositors (e.g., filling nozzles) onto starch lined trays (“mogul boards”). The mogul boards allow the cooked candy to firm and take on the shape of the tray mold, to produce a series of shaped gummy candies. In one implementation, the depositors are timed to automatically deliver the exact amount of candy needed to fill the trays as the mogul boards are passed under the depositors. In other implementations, the coloring, flavoring, and acids added to the cooked gummy candy at step 122, may be added to the candy in the depositor.

A mogul is called a starch molding machine because starch is a main component of the machine. In this machine, starch has three primary purposes. First, it prevents the gummy candy stock from sticking to the mogul boards, which allows for easy removal and handling. Second, starch holds the gummy candy in place during the drying, cooling, and setting processes. Finally, starch absorbs moisture from the candies, giving them the proper texture.

In some cases, the starch used to coat the mogul boards may include recycled starch; wet starch that falls away from the candies when they are removed from the mogul boards. The re-used starch may be recycled to a starch dryer where the starch is sifted and dried. After the starch is died, it may then be cooled in a starch cooler. The cooled starch may be sifted a second time and returned to the mogul where it may be re-circulated once again, through the same process. The recycled starch may then be sprayed evenly on the mogul board, where the cooked candy may then be deposited onto mogul boards coated with the recycled starch.

After the cooked candy is deposited onto the mogul boards, the mogul boards may be stacked, then removed from the stack (one-by-one) by a conveyor belt, and finally placed in a temperature and humidity controlled curing room, where the candy sits and cools (i.e., is cured) for approximately 24 hours to 48 hours (step 132). However, the curing time for the cooked candy may vary based on binding agent and the temperature and humidity of the curing room. Proper curing time is necessary to solidify, or set the gummy product to ensure ease of packaging without breakage and proper yield. In one implementation, the candy may be cured in a curing room with approximately 15% to 25% humidity.

After curing, the gummy candies, firmed and having proper texture, may be moved to a section of the mogul called the starch buck. In the starch buck, the mogul boards are inverted and the gummy candy is dumped into a tumbler machine at step 134. In one implementation, the tumbler may include a 2,000 gallon rotating drum or, in other implementations, a vibrating metal sieve. In the tumbler, the gummies may be tumbled together to remove any excess starch that adheres to the gummy candies. Once the starch is removed, the gummies may become sticky, so the gummies may be coated with a polishing compound to prevent the cooked candies from sticking together. Depending on the desired finished product or preferences, the gummies may be polished with fractionated coconut oil, linseed oil, sunflower oil, bees wax, carnauba wax, mineral oil, or any other suitable food grade oil or combination thereof. In other implementations, the gummies may be sanded with sugar or a sugar substitute in a drum.

After the gummies are coated, they may be placed on a cooling belt (e.g., a conveyor belt) and transported to an inspection station at step 136. At step 136, the gummy candies are placed on an inspection belt where the candies are inspected for food safety and proper organoleptic effects. For example, on the inspection belt the gummy candies may be passed by a detector or x-ray to insure that no particulate or other foreign material has been deposited into the candy during the depositing stage.

Moving on to step 138, once the candy passes inspection, the finished gummy product is packaged for distribution.

The disclosure above only describes one implementation of a method of manufacturing a delivery system of the present invention. Other methods and implementations may be used to manufacture delivery systems in accordance with the present invention.

### EXAMPLES

The following examples describe particular formulations and concentrations thereof for preparing chewable supplements of the present invention. Chewable supplements of the present invention may include non-organic and/or organic compositions. For example, in one implementation, the chewable supplement may include a non-organic or an organic gummy candy. While the process of manufacturing a non-organic gummy and an organic gummy are similar, as described above, the formulations for the two systems differ, as explained in more detail below.

### Non-organic Supplement

In one implementation, the delivery system of the present invention may include a non-organic gummy. For example, a non-organic chewable supplement in accordance with the present invention may be prepared using the following formula:

TABLE B
NON-ORGANIC GUMMY FORMULA

| Ingredients | Content (by Weight) |
|---|---|
| Water | 6.0% |
| Lactic acid | 1.0% |
| Citric acid | 1.0% |
| Natural cane juice | 35.0% |
| Glucose syrup | 45.0% |
| Gelatin | 7.0% |
| Blend of omega fatty acids | 3.0% |
| Flavoring | 1.5% |
| Coloring | 0.5% |

In this example, about 93 lbs of warm water may be mixed with about 7 lbs of gelatin compound in the mixing tank to form 100 lbs of gelling compound having a homogeneous 93/7 blend of water and gelatin. In one implementation, the gelatin ingredient may include a pork-based GELITA® Gelatine product, or any other suitable gelatin ingredient. To stabilize the gelling compound, about 0.1% to 10% of sodium bisulfate by weight may be added to the compound to temper its pH to about 3.5.

In the mixing weigh vessel, the gelling compound may be mixed with about 6 lbs of water, 35 lbs of natural cane juice, and 45 lbs of glucose syrup to form the candy slurry mixture. In addition to sweeteners, about 2 lbs to 3 lbs of a propriety blend of omega fatty acids may be added to the candy slurry mixture at step 114 (FIG. 1). In one implementation, the propriety blend of omega fatty acids may include about 163 mg of chia oil (ALA), 33 mg of corn oil (LA), and 65 mg of olive oil (OA).

Next, the candy slurry may be heated to a temperature of about 180° F. prior to being passed through the storage buffer tank, to the static cooker. In the static cooker, the candy slurry may be heated to a temperature of about 240° F. to 245° F., dehydrating the slurry to a brix of about 78.

After the candy is cooked, the cooked candy is sent to the vacuum, where the candy may be further dehydrated to a brix of about 80. After leaving the vacuum, the cooked candy is placed in the dosier where about 1.5% of cranberry and orange flavoring by weight and about 0.5% of black carrot juice coloring by weight may be added to the cooked candy. To balance the flavoring, about 0.1% citric acid by weight and about 0.1% lactic acid by weight may be added to the cooked candy.

After adding the flavoring and coloring, the cooked candy may be deposited into the mogul machine and then cured. After the candies are cured, they may be added to a tumbling drum to break off any starch that may be remaining on the candies. As the candies are being tumbled, about 1% fractionated coconut oil by weight and about 1% carnauba wax by weight may be poured into the drum to coat the candies to prevent them from sticking together.

After the candies are coated, they may be inspected to validate that the finished product meets the label requirements, and then packaged.

### Organic Supplement

In another implementation, the delivery system of the present invention may include an organic gummy. To create an organic gummy, the ingredients used to form the drug must meet the requirements for organic certification. These ingredients may include, but not be limited to, organic evaporated cane juice, organic tapioca syrup, organic grape juice, citric acid, lactic acid, sodium citrate, natural color (e.g., black carrot juice concentrate, annatto, turmeric, purple berry concentrate) and natural flavor (e.g., strawberry, orange, pineapple, grape), and a proprietary blend of vitamins, minerals and other functional ingredients.

For example, an organic chewable supplement in accordance with the present invention may be prepared using the following formula:

TABLE C
ORGANIC GUMMY FORMULA

| Ingredients | Content (by Weight) |
|---|---|
| Water | 9.5% |
| Lactic acid | 1.0% |
| Citric acid | 1.0% |
| Organic evaporated cane juice | 35.0% |
| Organic tapioca syrup | 46.0% |
| Pectin | 2.0% |
| Chia oil (ALA) | 3.0% |
| Natural flavoring | 1.5% |
| Natural coloring | 1.0% |

In this example, about 98 lbs of warm water may be mixed with about 2 lbs of pectin in the mixing tank, to form 100 lbs of gelling compound having a homogeneous 98/2 blend of water and pectin. In one implementation, the pectin ingredient may include UNIPECTIN® HM-pectin products, or any other suitable pectin ingredient. To stabilize the gelling compound, about 0.1% to 10% of sodium bisulfate by weight may be added to the compound to reduce its pH to about 3.5.

In the mixing weigh vessel, the gelling compound may be mixed with about 6 lbs of water, 35 lbs of organic evaporated cane juice, and 46 lbs of organic tapioca syrup to form the candy slurry mixture. In addition to sweeteners, about 2 lbs to 3 lbs of chia oil may be added to the candy slurry at step 114 (FIG. 1).

Next, the candy slurry may be heated to a temperature of about 180° F. prior to being passed through the storage buffer tank, to the static cooker. In the static cooker, the candy slurry may be heated to a temperature of about 240° F. to 245° F., dehydrating the slurry to a brix of about 78.

After the candy is cooked, the cooked candy is sent to the vacuum, where the candy may be further dehydrated to a brix of about 80. After leaving the vacuum, the cooked candy is placed in the dosier where about 1.5% of orange and cherry flavoring by weight and about 1% of annatto and turmeric coloring by weight may be added to the cooked candy. To balance the flavoring, about 0.1% citric acid by weight and about 0.1% lactic acid by weight may be added to the cooked candy.

After adding the flavoring and coloring, the cooked candy may be deposited into the mogul machine and then cured. After the candies are cured, they may be added to a tumbling drum to break off any starch that may be remaining on the candies. As the candies are being tumbled, about 1% fractionated coconut oil by weight and about 1% carnauba wax by weight may be poured into the drum to coat the candies to prevent them from sticking together.

After the candies are coated, they may be inspected to validate that the finished product meets the label requirements, and then packaged.

### Starch-Based Supplement

In another implementation, the delivery system of the present invention may include a pure starch-based gummy For example, a starch-based chewable supplement in accordance with the present invention may be prepared using the following formula:

TABLE D
STARCH-BASED GUMMY FORMULA

| Ingredients | Content (by Weight) |
|---|---|
| Water | 6.5% |
| Lactic acid | 1.0% |
| Citric acid | 1.0% |
| Sucrose | 34.0% |
| Corn syrup | 45.0% |
| Rice starch | 7.0% |
| Chia oil (ALA)/olive oil (OA) | 3.0% |
| Natural flavoring | 1.5% |
| Natural colorant | 1.0% |

In this example, about 93 lbs of warm water may be mixed with about 7 lbs of rice starch in the mixing tank, to form 100 lbs of gelling compound having a homogeneous 93/7 blend of water and starch.

In the mixing weigh vessel, the gelling compound may be mixed with about 6 lbs of water, 34 lbs of natural cane juice, and 45 lbs of corn syrup to form the candy slurry. In addition to sweeteners, about 2 lbs to 3 lbs of a chia oil/olive oil compound may be added to the candy slurry at step 114 (FIG. 1).

Next, the candy slurry may be heated to a temperature of about 180° F. prior to being passed through the storage buffer tank, to the static cooker. In the static cooker, the candy slurry may be heated to a temperature of about 240° F. to 245° F., dehydrating the slurry to a brix of about 78.

After the candy is cooked, the cooked candy is sent to the vacuum, where the candy may be further dehydrated to a brix of about 80. After leaving the vacuum, the cooked candy is placed in the dosier where about 1.5% of orange and cherry flavoring by weight and about 1% of annatto and turmeric coloring by weight may be added to the cooked candy. To balance the flavoring, about 0.1% citric acid by weight and about 0.1% lactic acid by weight may be added to the cooked candy.

After adding the flavoring and coloring, the cooked candy may be deposited into the mogul machine and then cured. After the candies are cured, they may be added to a tumbling drum to break off any starch that may be remaining on the candies. As the candies are being tumbled, about 1% fractionated coconut oil by weight and about 1% carnauba wax by weight may be poured into the drum to coat the candies to prevent them from sticking together.

After the candies are coated, they may be inspected to validate that the finished product meets the label requirements, and then packaged.

In accordance with the teachings of the present invention, starch-based gummies provide an additional benefit over traditional gelatin-based gummies, or any other candy made from a thermoreversible gelling agent. In particular, because gelatin liquefies when heat is applied, gelatin-based gummies frequently melt when they are exposed to high temperatures during storage and transport. But starch is more stable than gelatin in high temperatures. This is because the semi-crystalline structure of starches do not fully recover once a starch is gelatinized (i.e., becomes a gel when cooked in water) and then cooled, so the starch becomes a thickened paste. If additional heat is applied to the thickened paste, the starch will not liquefy since the starch granules swell and burst during the gelatinization process. Thus, starch-based gummies may retain their gummy shape under high temperature without melting. This is ideal for gummies that are exposed to high temperatures during storage and transport. As used herein, “thermoreversible gelling agents” refer to gels or compounds that melt or liquefy when heated.

The examples provided above are for illustrative purposes only. Formulations for chewable compositions of the present invention may vary based on the desired dosage of active ingredients and the amount of additives, sweeteners, and coloring added to the drug composition.

While implementations of the invention have been described with reference to a gummy delivery system, the invention is not limited to this application and may be readily used for any chewable or digestible composition. For example, implementations of the invention may also be employed in organic, vegetarian or non-vegetarian, tablets, capsules, or solid candies. For purposes of the present invention, the term “vegetarian” refers to a product or composition that does not contain any animal ingredients or by-products. The present invention may also apply to other forms of candies such as jelly beans or caramel-based candies. Further, while the dimensions of the holding and mixing vessels are provided herein by way of example only, the actual dimensions of these vessels may vary based on the amount of gelling compound and candy slurry produced in a given time period (e.g., per day).

The foregoing description of implementations has been presented for purposes of illustration and description. It is not exhaustive and does not limit the claimed invention to the precise form disclosed. Modifications and variations are possible in light of the above description or may be acquired from practicing the invention. The claims and their equivalents define the scope of the invention.

What is claimed is:

1. A chewable composition comprising a plant-based omega fatty acid.

2. The chewable composition of claim 1 further comprising a binding agent and a sweetener.

3. The chewable composition of claim 1, wherein the omega fatty acid is an α-linolenic acid derived from a plant based source.

4. The chewable composition of claim 1, wherein the omega fatty acid is docosahexaenoic acid derived from a plant-based source.

5. The chewable composition of claim 1, wherein the omega fatty acid is linoleic acid derived from a plant-based source.

6. The chewable composition of claim 1, wherein the omega fatty acid is oleic acid derived from a plant-based source.

7. The chewable composition of claim 1, wherein the omega fatty acid includes any combination of omega fatty acids derived from a plant-based source.

8. The chewable composition of claim 2, wherein the binding agent is a gelatin base.

9. The chewable composition of claim 2, wherein the binding agent is a pectin base.

10. The chewable composition of claim 2, wherein the binding agent is a starch base.

11. The chewable composition of claim 2, wherein the binding agent is a carrageenan base.

12. The chewable composition of claim 1 further comprising any combination of supplements selected from the group consisting of vitamins, minerals, nutraceuticals, probiotics, prebiotics, herbs, fibers, antioxidants, amino acids, digestive enzymes, dietary supplements, or any other health promoting ingredient.

13. The chewable composition of claim 2 wherein the binding agent and sweetener are organic compliant.

14. The chewable composition of claim 13 wherein the composition qualifies as a composition capable of being certified as organic or vegetarian.

15. A delivery system for health supplements comprising:
a plant-based omega fatty acid; and
a digestible delivery vehicle containing the plant-based omega fatty acid.

16. The delivery system of claim 15, wherein the digestible delivery vehicle includes a binding agent and a sweetener.

17. The delivery system of claim 16, wherein the digestible delivery vehicle is a chewy gummy candy.

18. The delivery system of claim 15, wherein the digestible delivery vehicle is a vehicle selected from the group consisting of tablets, capsules, solid or chewable candies.

19. The delivery system of claim 15, wherein the omega fatty acid is an α-linolenic acid derived from a plant based source.

20. The delivery system of claim 15, wherein the omega fatty acid is docosahexaenoic acid derived from a plant-based source.

21. The delivery system of claim 15, wherein the omega fatty acid is linoleic acid derived from a plant-based source.

22. The delivery system of claim 15, wherein the omega fatty acid is oleic acid derived from a plant-based source.

23. The delivery system of claim 15, wherein the omega fatty acid includes any combination of omega fatty acids derived from a plant-based source.

24. The delivery system of claim 16, wherein the binding agent is a gelatin base.

25. The delivery system of claim 16, wherein the binding agent is a pectin base.

26. The delivery system of claim 16, wherein the binding agent is a starch base.

27. The delivery system of claim 16, wherein the binding agent is a carrageenan base.

28. The delivery system of claim 15 further comprising any combination of supplements selected from the group consisting of vitamins, minerals, nutraceuticals, probiotics, prebiotics, herbs, fibers, antioxidants, amino acids, digestive enzymes, dietary supplements, or any other health promoting ingredient.

29. The chewable composition of claim 16 wherein the binding agent and sweetener are organic compliant.

30. The chewable composition of claim 29 wherein the composition qualifies as a composition capable of being certified as organic or vegetarian.

31. A chewable composition comprising:
a gelatin binding agent;
a sweetener; and
a plant-based omega fatty acid; and
at least one vitamin supplement.

32. The chewable composition of claim 31 wherein the omega fatty acid includes any combination of omega fatty acids derived from a plant-based source.

33. The chewable composition of claim 31 wherein the vitamin supplement comprises any combination of any combination of vitamins, minerals, nutraceuticals, probiotics, prebiotics, herbs, fibers, antioxidants, amino acids, digestive enzymes, dietary supplements, or any other health promoting ingredient.

34. A chewable composition comprising:
a binding agent that is organic compliant;
a sweetener that is organic compliant; and
a plant-based omega fatty acid; and
at least one vitamin supplement.

35. The chewable composition of claim 34 wherein the omega fatty acid includes any combination of omega fatty acids derived from a plant-based source.

36. The chewable composition of claim 34 wherein the vitamin supplement comprises any combination of any supplements selected from the group consisting of vitamins, minerals, nutraceuticals, probiotics, prebiotics, herbs, fibers, antioxidants, amino acids, digestive enzymes, dietary supplements, or any other health promoting ingredient.

37. The chewable composition of claim 34 wherein the composition qualifies as a composition capable of being certified as organic or vegetarian.
